(19) United States
(12) Patent Application Publication
(10) Pub. No.: US 2016/0367559 A1
(43) Pub. Date: Dec. 22, 2016

(54) Time Released Caffeine

(71) Applicant: Richard Brockman, New York, NY (US)

(72) Inventors: Richard Brockman, New York, NY (US); Jack M. Gorman, Riverdale, NY (US)

(21) Appl. No.: 15/135,242

(22) Filed: Apr. 21, 2016

Related U.S. Application Data
(60) Provisional application No. 62/182,194, filed on Jun. 19, 2015.

(51) Int. Cl. A61K 31/522; A61K 9/20; A61K 9/48; A61K 9/00

(52) U.S. Cl. CPC A61K 31/522; A61K 9/0053; A61K 9/20; A61K 9/48

(57) ABSTRACT

A pill that increases the availability of caffeine in the bloodstream of a user by delaying release of the drug from the stomach for the treatment of neurological, cognitive, dementia, anxiety, panic, depression, eating, bi-polar, attention deficit disorder, attention deficit hyperactivity disorder, obsessive compulsive disorder, and sleep related disorders. The delay can be enacted by several known means for creating extended release tablets including, polymer based tablets, microencapsulation, and matrix embedding, such that the caffeine is administered to a patient in an effective amount in a time released pill form. This requires fewer administrations of the drug while creating a smoother user experience as the build up and removal of the drug in the body is more gradual.

### CROSS-REFERENCE TO RELATED APPLICATION

This application claims the priority of U.S. Provisional Patent Application No. 62/182,194, filed Jun. 19, 2015 and entitled, “Time Released Caffeine,” which is incorporated by reference herein.

### FIELD OF THE INVENTION

The present invention relates generally to the field of medical treatment, and more particularly to increasing the availability of the active drug-like ingredient in coffee or caffeine in the bloodstream of a user to allow the same to be easily ingested and to thereby provide longer efficacy for the user and to also provide a smoother and less drastic time for activation of the effects as well as a smoother time for the effect of the same to tail off, all to the advantage of the user. The present invention is intended to treat neurological, cognitive, dementia, anxiety, panic, depression, eating, bi-polar, attention deficit disorder, attention deficit hyperactivity disorder, obsessive compulsive disorder, and sleep related disorders as a single treatment modality or as an adjuvant. Consistent with the present invention, the active ingredient of coffee or caffeine is physically contained within, preferably, time release capsules, caplets, or similar time release of active ingredient drug-like delivery devices/systems. In this manner, the same can be directly ingested, rather than being severely diluted with water in a cup of coffee. This will allow the active ingredient to more smoothly come into play, allow the caffeine's activity to remain “active” for longer time periods, and then, when the activity of the caffeine is finished, the ramp down of its effect will also be far smoother in transition than that currently accomplished and experienced by coffee cup drinkers. In addition to the above, the present invention reduces the adverse side effects felt by users who ingest caffeine.

The availability of the active caffeine ingredient in the bloodstream of a user can be enacted by several known means for creating extended release tablets including, polymer based tablets, micro-encapsulation, and matrix embedding, such that the caffeine is administered to a patient in a time released pill form. This requires fewer administrations of the drug, provides the same in a less diluted and more active manner (not a component of a cup of coffee) while creating a smoother user experience as the build up and removal of the drug in the body is more gradual. The invention can also be used as an adjuvant to antidepressants rather than caffeine but the principal thrust of the present invention relates to time release of a more purified form of caffeine by encasing the same in a time release capsule, caplet, etc. The invention can also be used to extend the availability of caffeine in the bloodstream of a user so it can be used as an anti-depressant in its own right, or as an adjunct with other anti-depressant medications.

### BACKGROUND OF THE INVENTION

In North America, the overwhelming majority of adults consume caffeine daily. Many consume three to four cups of coffee a day, or the equivalent of 300 milligrams of caffeine, the key active ingredient to the cup of coffee, to maintain caffeine levels in the body throughout the day. This is because caffeine is relied on by many for alertness and productivity. It is also generally considered safe with few adverse side effects. Moreover, the sustained release formulation will be more easily tolerated by the user.

Caffeine is believed to have other beneficial uses as well including improving body coordination, endurance, and mental performance. With the wide range of benefits and few seeming drawbacks, caffeine is one of the most popular “drugs” today. The ingestion and administration of caffeine to everyday consumers is not perfect however. For one, the vast majority of caffeine users get their caffeine through cups of coffee. Because coffee contains a small dose of caffeine in comparison to large amounts of water, the caffeine's ingestion requires large amounts of water intake. The caffeine of the coffee is only active for a short period of time and then its half life reduces its effectiveness. Indeed, the body removes its efficacy within hours; users must continuously refill their cups of coffee during the day to feel the effects over a longer period of time. Refilling is problematic as it takes valuable time and resources. Caffeine-laced or pure pills may allow for higher doses to be ingested but those higher doses are often unpractical as they may cause uncomfortable side effects such as anxiety, jitters, and result in the difficulty of relaxing and sleeping. The higher doses also do not affect the half-life of the medication; although and because they consist of more caffeine their effects will last longer. It would thus be advantageous to provide caffeine in a form that provides healthy doses for an extended period of time that does not require refilling or re-administration of the drug. It would be advantageous, too, to provide the ingestion of this beneficial substance with an extended availability in the bloodstream of a user so that comfortable dosages remain active for a longer period of time.

Another problem with caffeine consumption is that, although caffeine may take up to thirty minutes to become effective, its presence, and subsequent removal, in a person's system can feel quite sudden. This causes quick jolts of energy as well as seemingly as quick declines. It thus would be advantageous to provide caffeine in a form that provides a more gradual build up in the body of its utility and a similarly equally gradual removal of its effectiveness from the body.

The current invention provides for an extended time release caffeine-based pill or tablet used to treat neurological, cognitive, dementia, anxiety, panic, depression, eating, bi-polar, attention deficit disorder, attention deficit hyperactivity disorder, obsessive compulsive disorder, and sleep related disorders. The present invention slowly releases doses of caffeine into the body over several hours and extends the availability of the drug in the bloodstream of a user while, at the same time, ensuring that the impact of the caffeine in the system is not too drastic and the drop off of the effectiveness of the caffeine is similarly not too steep. The extended time release caffeine based pills works to gradually introduce and remove caffeine in the body, making its effect to come into play and its fade from utility more gradual. In addition to the uses identified above, these pills can be used to enhance the effectiveness of antidepressant medications. By making their action last longer, these pills can be used for their anti-depressant effects when used alone or in conjunction with other anti-depressant medications.

Most currently available anti-depressants require a minimum of ten (10)-fourteen (14) days before there is any improvement in mood. They also have significant side effects including sedation and weight gain. The current invention, long acting caffeine, has a much more rapid onset of action with improvement in mood usually occurring in less than an hour. There is also an increase of alertness and suppression of appetite associated with caffeine. Thus the use of the present invention with other antidepressants to hasten therapeutic improvement and to diminish the common side-effects of sedation and weight gain often seen with most current anti-depressants, are additional benefits anticipated.

### DESCRIPTION OF THE PRIOR ART

To the Applicant's knowledge, no prior art exists which discloses an extended time release caffeine-based pill or tablet used for the treatment of neurological, cognitive, anxiety, panic, depression, eating, bi-polar, attention deficit disorder, attention deficit hyperactivity disorder, obsessive compulsive disorder, and sleep related disorders. In addition to these uses, to the Applicant's knowledge, no prior art exists which discloses extended release pills caplets, tablets based on caffeine or other similar products resulting a long lasting but healthy levels of caffeine in the body with smoother times to active effect and smoother times to drop off of effect. Thus it is considered advantageous to provide such a time-release device for administering a long lasting but comfortable dose of caffeine to a user for the treatment of neurological, cognitive, dementia, anxiety, panic, depression, eating, bi-polar, attention deficit disorder, attention deficit hyperactivity disorder, obsessive compulsive disorder, and sleep related disorders over an extended period of time.

Prior to submitting the present application, Applicant performed a patent search which revealed very little in the art thereby amplifying the novelty to the present invention. U.S. Pat. Nos. 5,744,164 and 5,700,484 (hereinafter referred to as the “Chauffard patents”) by Francoise Chauffard, discloses a composition and method for increasing the alertness of an individual through the administration of a stimulant such as eanthine or an xanthine derivative and preferably caffeine. The Chauffard patent fails to disclose a time released caffeine pill used for the treatment of neurological, cognitive, dementia, anxiety/panic/depression, eating, bi-polar, attention deficit disorder, attention deficit hyperactivity disorder, obsessive compulsive, and sleep related disorders.

Chinese Patent Application Number CN20091102031 (hereinafter referred to as the “Xiaomei Guo patent”) by Xiaomei Guo, et al, discloses a slow release micro pill preparation, which comprises the following components in percentage by weight: 50-80% of caffeine, 15-30% of empty pill core, 1-4% of binder, 10-20% of slow-release material of 5-20% of antiadherent. By taking the caffeine slow-release micro pill preparation once a day, effective blood concentration can be maintained for 8-10 hours. The Xiamei Guo patent claims to improve awareness of the user for 8-10 hours. The Xiaomei Guo patent fails to disclose a time released caffeine pill used for the treatment of neurological, cognitive, dementia, anxiety/panic/depression, eating, bi-polar, attention deficit disorder, attention deficit hyperactivity disorder, obsessive compulsive, and sleep related disorders.

Chinese Patent Application Number CN102648914 (hereinafter referred to as the “Fan Minhua patent”) by Fan Minhua, et al., discloses the use of caffeine in preparation of medicine for treating Senile Dementia. The Fan Minhua patent fails to disclose a time released caffeine pill used for the treatment of anxiety/panic/depression, eating, bi-polar, attention deficit disorder, attention deficit hyperactivity disorder, obsessive compulsive, and sleep related disorders.

PCT Patent Application Number PCT/US2014/040916 (hereinafter referred to as the “Upugalla patent”) by Narender R. Upagalla, et al., relates to the administration of a caffeine-containing dosage form providing an initial immediate release of caffeine, followed by a controlled release of caffeine to maintain caffeine levels in the body over time. The Upagalla patent application fails to disclose a time released caffeine pill used for the treatment of neurological, cognitive, dementia, anxiety/panic/depression, eating, bi-polar, attention deficit disorder, attention deficit hyperactivity disorder, obsessive compulsive, and sleep related disorders.

### SUMMARY OF THE INVENTION

The present invention generally relates to a method for treating a disease or disorder responsive to the administration of caffeine. This method comprises of identifying a patient suffering from a disease or disorder and administering a therapeutically effective amount of caffeine to the patient. The caffeine administered to the patient is typically ingested orally via a pill, tablet or caplet that uses time-release technology, also known as slow-release and extended-release, to increase the length of time for the caffeine's effectiveness and makes the effect and wearing off feel more gradual to a user than drinking cups of coffee. The present invention is administered as a treatment for neurological, cognitive, dementia, anxiety, panic, depression, eating, bi-polar, attention deficit disorder, attention deficit hyperactivity disorder, obsessive compulsive disorder, and sleep related disorders.

The invention may use any number of time release methods for pills. For example, the caffeine may be embedded in a matrix of insoluble substances, enclosed in a polymer based tablet, or micro-encapsulated around a microsphere. Each of these somewhat conventional methods will extend the availability of the drug in the blood, thereby extending the metabolism rate of the drug. As a result, the wholesale consumption of the entire amount of caffeine in the pill or capsule is delayed. Time release technology, for caffeine, is expected to increase the availability of the caffeine in the bloodstream of a user and, at the same time, ensure that the impact and effects of the caffeine be more gradual to the user and its wearing off be similarly more gradual. This is believed to be beneficial to the user on many levels, which include, but are not limited to the following: the treatment of neurological, cognitive, dementia, anxiety/panic/depression, eating, bi-polar, attention deficit disorder, attention deficit hyperactivity disorder, obsessive compulsive, and sleep related disorders. The caffeine dosage used to treat the above described; diseases and/or disorders is about 125 to 600 mg; however, said dosage may range depending on the weight, age and overall tolerance one has to caffeine. For example, the following dosages of caffeine may be administered to a patient to treat neurological, cognitive, dementia, anxiety, panic, depression, eating, bi-polar, attention deficit disorder, attention deficit hyperactivity disorder, obsessive compulsive disorder, and sleep related disorders as a single treatment modality or as an adjuvant:

Dosage Strength and Range (mg)

125-200
125-300
125-400
125-500
125-600
200-300
200-400
200-500
200-600
300-400
300-500
300-600
400-500
400-600
500-600

Because a user will feel the effect of caffeine until only a small minimum or threshold amount remains unmetabolized, extending the amount of time the drug is in the blood and therefore the amount of time for its effectiveness in the body of a user. Typically, caffeine has a half life of about four (4) to six (6) hours in the body. Using the present invention, the availability of caffeine can be extended to more than double that so that, for the same amount of active caffeine, the availability of caffeine in the same body will be increased to about six (6) to eight (8) hours. Moreover, using the present invention, the caffeine dosage is uniformly released over a period of eight (8) to ten (10) hours. Because the drug is more slowly released into the blood (a consequence of the time release of the active caffeine by time release technology) it will also more slowly reach noticeable and active levels as well as more slowly dissipate. As a result the onset and fading of caffeine's effects will feel more gradual and natural to a user. This will result in less “craving” for the same, in less jitters and quick-movements during ramp up and in less depression and other caffeine withdrawal symptoms during the wearing off of the effectiveness of the effects of the caffeine.

Caffeine is a very widely used “drug” with many perceived benefits including mood regulation, increased productivity, alertness, and coordination. The present invention allows users to gain these benefits for an extended period of time and without sudden changes in anxiety and feeling. Moreover, as previously discussed, the present invention may be used for the treatment of neurological, cognitive, dementia, anxiety/panic/depression, eating, bi-polar, attention deficit disorder, attention deficit hyperactivity disorder, obsessive compulsive, and sleep related disorders.

These and other objects, features, and advantages of the present invention will become apparent from the following detailed description of illustrative embodiments thereof.

### DETAILED DESCRIPTION OF THE INVENTION

Various aspects and embodiments of the present invention will now be described in detail. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the disclosure to the precise form disclosed. These embodiments are chosen and described to best explain the principle of the invention and its application and practical use and to enable others skilled in the art to best utilize the invention.

Caffeine is one of the most widely used substances today to promote awareness, alertness, concentration, and focus. It is consumed by roughly ninety percent of American adults on a daily basis. Indeed, many do not consider themselves fully awake until and unless they have their morning cup of coffee. Many, too, have intra-day cups of coffee to obtain the continued effect of caffeine on their body throughout the workday. Caffeine also has many uses ranging from mood stability and increased alertness to improved muscle coordination. Its effect is usually rapid and this can cause shaking, too quickness of action, speaking, and excessive perspiration. The results of caffeine will also dissipate quickly however, causing users to continuously consume more caffeine during the day. To combat this drawback, the present invention incorporates caffeine into an extended release pill like device which slows absorption in the body and therefore makes the caffeine last longer, while also having the ramp up and ramp down of its effect on the body more smooth and less drastic, to the ultimate benefit of the user. In addition to the unique uses, the present invention facilitates the continuous and uniform release of at least about 10% by weight of the total amount of the ingested caffeine over a period of about eight (8) to ten (10) hours. The release of caffeine in this manner prevents users from suffering from caffeine highs and lows during the treatment period.

Oral medications and other drug-based substances taken by mouth as a consequence of the same being contained in a pill, caplet, or capsule are felt by the user generally after the medications are ingested. Few medications are intended to be consumed by placement on the tongue although it may be a quicker form to have the active ingredient gain access to the blood stream. Nevertheless, generally, now, a user swallows a pill, liquid, or other form of the medication where it enters the user's stomach and digestive track. The stomach will then begin to break down the pill and just as if it were food particles by use of enzymes to expose the medication. Blood vessels in the stomach and intestines absorb the medication- or a broken down usable form of the medication-and carry it via the blood to all parts of the body. It is at this point that a user will begin to feel the effects of the medication. The medication will reach key organs including the brain, muscles, and heart, all through the flow of blood to those organs and body parts. As the medication flows through the bloodstream, it also eventually reaches the liver. The liver removes impurities from the body including caffeine and breaks down the medication for elimination. Generally, the liver changes the active form of the drug, including caffeine, into a form that is no longer effective in the body for its original intended purpose. When enough of the medicine is removed and broken down, the user will then no longer be able to feel the effects of the drug. The amount of time it takes the body to break down half the amount of medication in the body is referenced to as the “half life.” It should be noted that, because the other half of the quantity of the medication remains at that point, a user will often still feel some effects of the medication after the half life duration, but the medication in the body will decrease, making the effects weaker and weaker (half life by half life duration of time) until it is completely removed unless a new dose is ingested. The broken down and non-usable form is then excreted from the body. This process is known as “metabolizing” or “metabolism.”

Caffeine as contained in a cup of coffee normally takes about thirty to forty-five minutes to reach the bloodstream and begin to take effect. Of course, many factors can come into play, strength of the coffee, quantity consumed, dilution, size and weight of the individual, the stomach's contents, etc. However, when the caffeine starts to take effect, at that point a user will feel more energized and alert. It can last in the body for several hours (depending again on various factors) but generally is thought of as having a half life of roughly four (4) to six (6) hours. With such a short half life with its positive effects being desired to last all day, sometime ten (10) hours or more, caffeine users must take several doses (via cups of coffee) over the course of a day as the body will continuously metabolize and remove the used drug from their system. Thus, it would be beneficial to ingest caffeine in some type of extended release form which would more smoothly release an efficacious and comfortable dose of the drug throughout the day. Time release caffeine pills which will satisfy the need by the user for the positive effects of the same throughout the work day and which will have a slower ramp up of the effects with a smoother and slower ramp down, when the caffeine is fully metabolized or below the threshold is desired. Extended releases pills are well known in the pharmaceutical industry for pharmaceuticals and drugs. In general, they reduce or slowly release the amount of a medication so that the user is maintained or titrated over time. The time release technology used in current pharmaceuticals can be used for the present invention. The time release is intended to provide a dose so that the medication is more slowly digested and therefore available for a longer period of time. There are several methods for extended release of a medication. One method is to embed the medication in a matrix of an insoluble substance such as acrylics, such that the drug will need to travel out of the matrix before it reaches the stomach to be absorbed. Inevitably, percentages of the dose will travel out of the matrix at various times making only a portion of the dose available, absorbed, and felt or effective at a time. Another method is to enclose the drug in a polymer based tablet. These tablets have a small hole on one end through which the drug can escape. On the other end is a porous membrane. Stomach acids slowly permeate the porous membrane, eventually reaching the medication and gradually pushing it out of the tablet into the stomach, where it will be absorbed and effective within the blood stream of a user. Another popular form of extended release pill is microencapsulated pills. Microencapsulation coats a medication in a slowly dissolvable material, such as gelatin. Either during ingestion or shortly after, the coating ruptures, exposing the drug to the stomach, where it is slowly digested and metabolized. Caffeine may be placed in any of the above time extended release pills or in others known in the market. Providing caffeine in such pills increases the amount of time that caffeine is available in the body, making its effects last longer. It also makes the effects of the drug, due to a wearing off of the caffeine (a consequence of the half life of the caffeine in the body) be gradual. This is highly desirous.

Such pills may also be used as antidepressants. An embodiment of the present invention comprises a time released dosage used for the treatment of neurological, cognitive, dementia, anxiety/panic/depression, eating, bi-polar, attention deficit disorder, attention deficit hyperactivity disorder, obsessive compulsive, and sleep related disorders. Medications in these forms will last throughout the day where the intended effects are felt longer and the ramp up and ramp down of the effect more gradual. This can greatly improve the comfort of a user as the medications will produce longer lasting, less sudden, and ultimately more natural relief from depression.

It should again be emphasized that the above-described embodiments of the invention are presented for purposes of illustration only. Many variations may be made in the particular arrangements shown. In addition, any simplifying assumptions made above in the course of describing the illustrative embodiments should also be viewed as exemplary rather than as requirements or limitations of the invention. Numerous other alternative embodiments within the scope of the appended claims will be readily apparent to those skilled in the art.

What is claimed is:

1. A method for treating a disease or disorder responsive to the administration of caffeine,
said method comprising the steps of:
identifying a patient suffering from said disease or disorder;
administering to said patient a dosage form comprising a therapeutically effective amount of caffeine;
wherein said caffeine is delivered in a time released solid dosage form.

2. The method of claim 1, wherein said dosage comprises a single dose.

3. The method of claim 1, wherein the time release solid dosage is initiated by either liposomes, proliposomes, microspheres, gels, prodrugs, cyclodextrins, nanoparticles, osmotic delivery, polymer-based, or microencapsulations.

4. The diseases or disorders of claim 1 comprising neurological, cognitive, dementia, anxiety, panic, depression, eating, bi-polar, attention deficit disorder, attention deficit hyperactivity disorder, obsessive compulsive disorder, and sleep related disorders.

5. The method of claim 1, wherein the amount of caffeine for each dosage is between about 125 mg to 600 mg.

6. The method of claim 1, wherein the amount of caffeine for each dosage is between about 200 mg to 500 mg.

7. The method of claim 1, wherein the amount of caffeine for each dosage is between about 300 mg to 400 mg.

8. The method of claim 5, wherein the time released dosage is capable of being released within 8-10 hours following oral administration.

9. The method of claim 6, wherein the time released dosage is capable of being released within 8-10 hours following oral administration.

10. The method of claim 7, wherein the time released dosage is capable of being released within 8-10 hours following oral administration.

11. The method of claim 1, wherein the caffeine is uniformly released over a period of 8-10 hours.

12. The method of claim 1, wherein at least about 10% by weight of the total amount of the caffeine orally ingested is continuously and uniformly released over a period of about 8 to 10 hours after ingestion.

13. The method of claim 5, wherein at least about 10% by weight of the total amount of the caffeine orally ingested is continuously and uniformly released over a period of about 8 to 10 hours after ingestion.

14. The method of claim 1, wherein said dosage form is suitable for oral administration.

15. The method of claim 1, wherein said solid dosage form is a tablet.

16. The method of claim 1, wherein said solid dosage form is a pill.

17. The method of claim 1, wherein said solid dosage form is a caplet.

18. The method of claim 15, wherein the administration of caffeine is used for the treatment of the treatment of neurological, cognitive, dementia, anxiety, panic, depression, eating, bi-polar, attention deficit disorder, attention deficit hyperactivity disorder, obsessive compulsive disorder, and sleep related disorders.

19. The method of claim 16, wherein the administration of caffeine is used for the treatment of the treatment of neurological, cognitive, dementia, anxiety, panic, depression, eating, bi-polar, attention deficit disorder, attention deficit hyperactivity disorder, obsessive compulsive disorder, and sleep related disorders.

20. The method of claim 17, wherein the administration of caffeine is used for the treatment of the treatment of neurological, cognitive, dementia, anxiety, panic, depression, eating, bi-polar, attention deficit disorder, attention deficit hyperactivity disorder, obsessive compulsive disorder, and sleep related disorders.
